(19) United States
(12) Patent Application Publication
(10) Pub. No.: US 2008/0318180 A1
(43) Pub. Date: Dec. 25, 2008

(54) DENTAL SCOPE

(75) Inventors: CHENG-I YANG, Jhonghe (TW); Ping-Tse Lin, Jhonghe (TW); Tin-Cheung Yau, Hong Kong (CN)

Correspondence Address: HEALTHY BIOTECH CORP. LTD., P.O. BOX 3-161, BANCIAO, 22099, TW

(73) Assignee: HEALTHY BIOTECH CORP. LTD., JHONGHE (TW)

(21) Appl. No.: 11/859,748

(22) Filed: Sep. 22, 2007

(30) Foreign Application Priority Data
Jun. 22, 2007 (TW) 096210176

(51) Int. Cl. A61B 1/24

(52) U.S. Cl. 433/31

(57) ABSTRACT

A dental scope including a stretchable rod, a fog-repellent reflection mirror group and a light module is disclosed. The fog repellent mirror group has a first fog-repellent reflection mirror connected to a distal end of the stretchable rod, and a second fog-repellent reflection mirror connected to a primal end of the stretchable rod. The light module has a spot light source, a battery and a switch disposed on the stretchable rod. The spot light source is employed to illuminate an interior of user's mouth.

This application claims the priority benefit of the Taiwan application serial No. 096210176 filed on Jun. 22, 2007.

### BACKGROUND OF THE INVENTION

1. Field of the Invention

The present invention relates to a dental scope for self inspecting oral health.

2. Description of Related Art

The conventional general oral and dental inspection scope comprises a handle and a mirror. The dentist holds the scope to inspect the interior of the patient's mouth and teeth under the spot light.

The mouth and teeth problems are usually identified by the patients themselves, and then the patients visit the dentist by spending the prime time for treatment. By regularly visiting the dentist in order to undergo conventional oral health inspection, some mouth and teeth problems are usually discovered. However, the patients themselves can not inspect their own oral health from time to time in order to timely identify the problem so that the damage to the teeth can be minimum or negligible. Therefore, it is highly desirable to improve the conventional oral health inspection scope such that the patients themselves may self inspect their interior of the mouth to timely identify the oral problems.

### SUMMARY OF THE INVENTION

An object of the present invention is to provide a dental inspection scope, which may be used by the users to self inspect interior of their mouth and teeth.

In accordance with the above object, the present invention provides a dental inspection scope comprising a light module for illuminating the mouth of the user and a reflective mirror group for reflecting the image of the interior of the user's mouth for the user to self inspect. Therefore, the oral health problem may be timely identified and fixed so that the damage may be minimal or negligible.

### BRIEF DESCRIPTION OF THE DRAWING

For a more complete understanding of the present invention, reference will now be made to the following detailed description of preferred embodiments taken in conjunction with the following accompanying drawings.

FIG. 1 is a perspective view of a dental scope according to a first embodiment of the present invention.

FIG. 2 is a perspective view of a dental scope according to a second embodiment of the present invention.

FIG. 3 is a perspective view of a dental scope according to a third embodiment C of the present invention.

FIG. 4 is a perspective view of a dental scope according to a forth embodiment of the present invention.

### DETAIL DESCRIPTION OF THE INVENTION

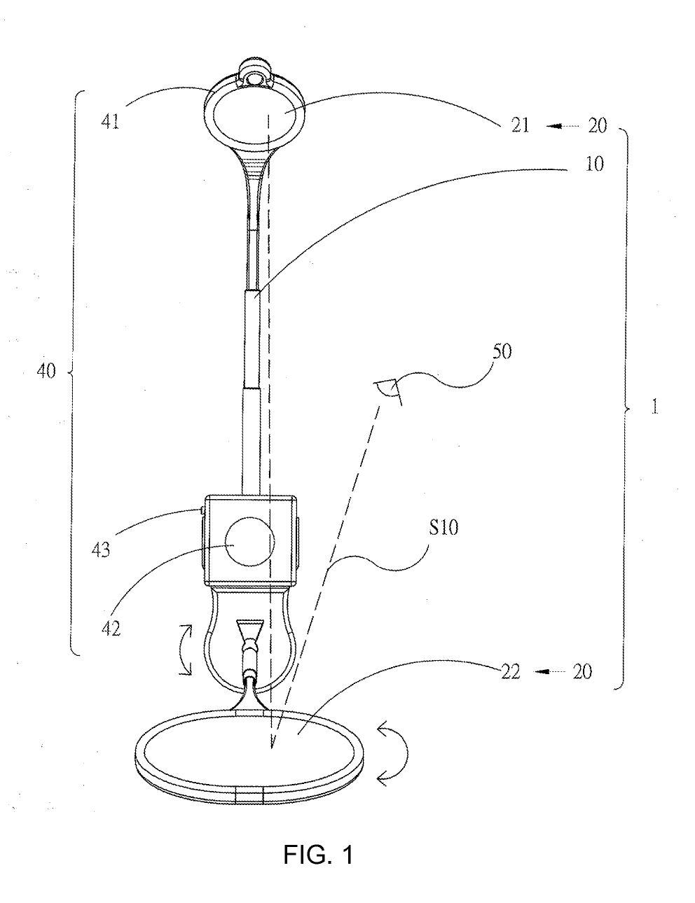

FIG. 1 is a perspective view of a dental scope according to a first embodiment of the present invention. Referring to FIG. 1, the dental scope 1 comprises a stretchable rod 10, a fog-repellent reflective mirror group 20 and a light module 40.

The fog-repellent reflection mirror group 20 comprises a first fog-repellent reflection mirror 21 and a second fog-repellent reflection mirror 22. The first fog-repellent reflection mirror 21 is connected to a flexible distal end of the stretchable rod 10, and the second fog-repellent reflection mirror 22 is connected to a primal end of the stretchable rod 10. The stretchable rod is rotatably detachable and has a spherical shaped head. The light module 40 comprises a spot light source 41, a battery 42 and a switch 43. The spot light source 41 is positioned on the first fog-repellent reflection mirror 21.

Hereinafter, the application of the dental scope, according to the first embodiment of the present invention, will be described. The stretchable rod 10 may be held and the length thereof may be adjusted as desired. Next, an angle of the first fog-repellent reflection mirror 21 relative to the stretchable rod 10 may be adjusted. Next, and an angle of the second fog-repellent reflection mirror 22 relative to the stretchable rod 10 may be adjusted such that the first fog-repellent reflection mirror 21 may be inserted into the user's mouth. Next, the switch 43 of the light module 40 is switched to turn on the spot light source 41 to illuminate the interior of the mouth so that the image of the interior of the mouth and teeth may be reflected on the first fog-repellent reflection mirror 21, which image in turn would be reflected on the second fog-repellent reflection mirror 22, and which image in turn would be reflected onto the user's eyes 50. The reflection path is indicated by S10. Thus, the dental scope of the present invention allows the user to self inspect the oral health.

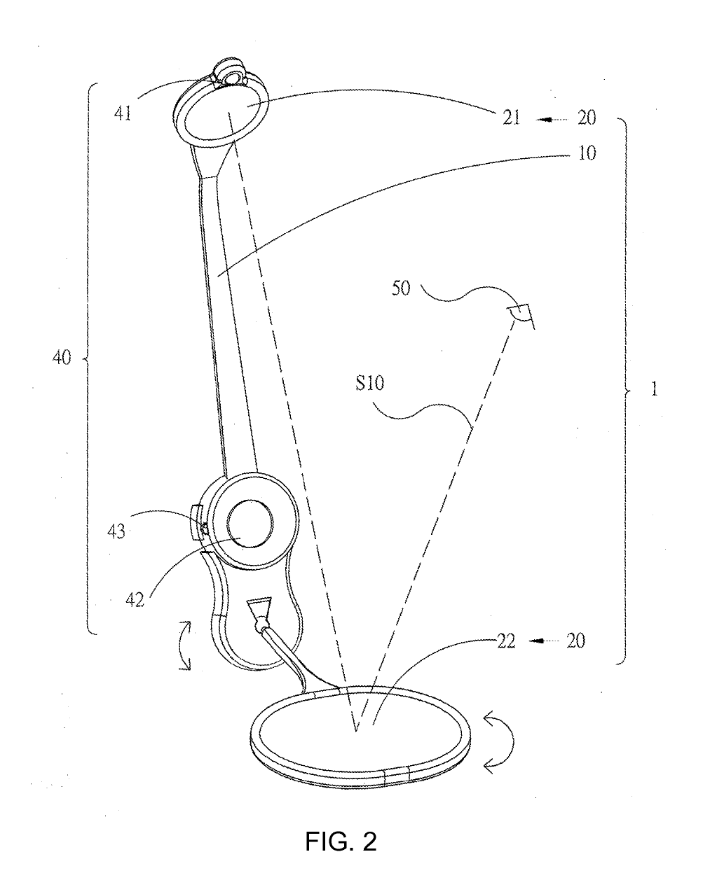

FIG. 2 is a perspective view of a dental scope according to a second embodiment of the present invention. Referring to FIG. 2, the dental scope 1 of the present invention comprises a stretchable rod 10 and a fog-repellent reflection mirror group 20 and a light module 40.

The fog-repellent reflection mirror group 20 comprises a first fog-repellent reflection mirror 21 and a second fog-repellent reflection mirror 22. The first fog-repellent reflection mirror 21 is fixed to a distal end of the stretchable rod 10, and the second fog-repellent reflection mirror 22 is connected to a primal end of the stretchable rod 10. The stretchable rod 10 is rotatably stretchable and detachable, and has a spherical shape head. The light module 40 comprises a spot light source 41, a battery 42 and a switch 43. The spot light source 41 is positioned on the first fog-repellent reflection mirror 21.

Hereinafter, the application of the dental scope, according to the second embodiment of the present invention, will be described. First, the stretchable rod 10 is held and a length thereof is adjusted as desired, and an angle of the second fog-repellent reflection mirror 22 is adjusted relative to the stretchable rod 10 such that the first fog-repellent reflection mirror 21 may be inserted into the user's mouth. Next, the switch 43 of the light module 40 is switched to turn on the spot light source 41 to illuminate the interior of the mouth so that the of the interior of the mouth and teeth may be reflected on the first fog-repellent reflection mirror 21, which image in turn would be reflected on the second fog-repellent reflection mirror 22, and which image in turn would be reflected to the user's eyes 50. The reflection path is indicated by S10. Thus, the dental scope of the present invention allows the user to self inspect the oral health.

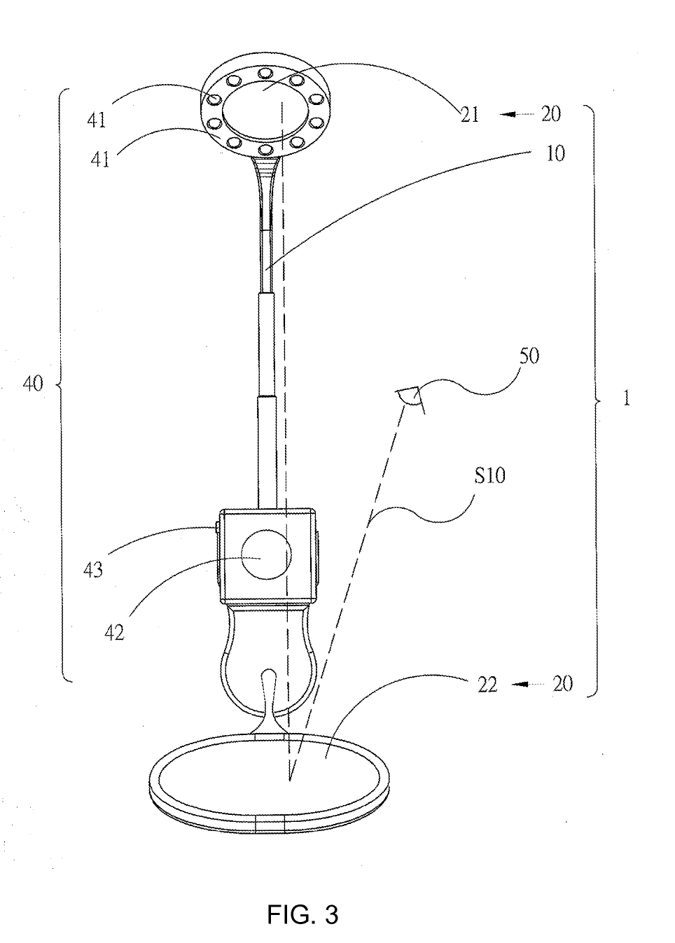

FIG. 3 is a perspective view of a dental scope according to a third embodiment of the present invention. Referring to FIG. 3, the dental scope 1 of the present invention comprises a stretchable rod 10 and a fog-repellent reflection mirror group 20 and a light module 40.

The fog-repellent reflection mirror group 20 comprises a first fog-repellent reflection mirror 21 and a second fog-repellent reflection mirror 22. The first fog-repellent reflection mirror 21 is connected to a flexible distal end of the stretchable rod 10, and the second fog-repellent reflection mirror 22 fixed to a primal end of the stretchable rod 10. The light module 40 comprises a plurality of spot light sources 41, a battery 42 and a switch 43. The spot light sources 41 are positioned on the first fog-repellent reflection mirror 21.

Hereinafter, the application of the dental scope, according to the third embodiment of the present invention, will be described. First, the stretchable rod 10 is held and then the length thereof is adjusted as desired and then an angle of the first fog-repellent reflection mirror 21 is adjusted relative to the stretchable rod 10 such that the first fog-repellent reflection mirror 21 may be inserted into the user's mouth. Next, the switch 43 of the light module 40 is switched to turn on the spot light sources 41 to illuminate the interior of the mouth so that the image of the mouth and teeth is reflected on the first fog-repellent reflection mirror 21, which image in turn would be reflected on the second fog-repellent reflection mirror 22, and which image in turn would be reflected to the user's eyes 50. The reflection path is indicated by S10. Thus, the dental scope of the present invention allows the user to self inspect the oral health.

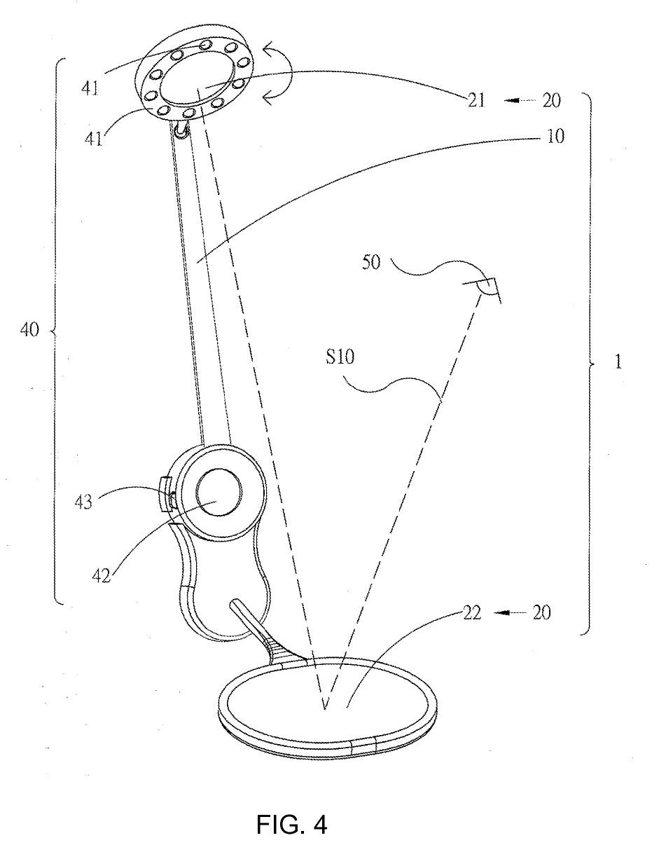

FIG. 4 is a perspective view of a dental scope according to a forth embodiment of the present invention. Referring to FIG. 4, the dental scope 1 of the present invention comprises a stretchable rod 10, a fog-repellent reflection mirror group 20 and a light module 40.

The fog-repellent reflection mirror group 20 comprises a first fog-repellent reflection mirror 21 and a second fog-repellent reflection mirror 22. The first fog-repellent reflection mirror 21 is fixed to a primal end of the stretchable rod 10. The stretchable rod 10 is rotatably stretchable and detachable, and has a spherical shaped head. The second fog-repellent reflection mirror 22 is connected to the distal end of the stretchable rod 10. The light module 40 comprises a plurality of spot light sources 41, a battery 42 and a switch 43. The spot light sources 41 are positioned on the first fog-repellent reflection mirror 21.

Hereinafter, the application of the dental scope, according to the forth embodiment of the present invention, will be described. First, the stretchable rod 10 is held and the length thereof is adjusted as desired. Next, an angle of the first fog-repellent reflection mirror 21 is adjusted relative to the stretchable rod 10. Next, an angle of the second fog-repellent reflection mirror 22 is adjusted relative to the stretchable rod 10 such that the first fog-repellent reflection mirror 21 may be inserted into the user's mouth. Next, the switch 43 of the light module 40 may be switched to turned on the spot light sources 41 to illuminate the interior of the mouth so that the image of the mouth and teeth may be reflected on the first fog-repellent reflection mirror 21, which image in turn is reflected on the second fog-repellent reflection mirror 22, and which image in turn is reflected to the user's eyes 50. The reflection path is indicated by S10. Thus, the dental scope of the present invention allows the user to self inspect the oral health.

While the invention has been described in conjunction with a specific best mode, it is to be understood that many alternatives, modifications, and variations will be apparent to those skilled in the art in light of the foregoing description. Accordingly, it is intended to embrace all such alternatives, modifications, and variations in which fall within the spirit and scope of the included claims. All matters set forth herein or shown in the accompanying drawings are to be interpreted in an illustrative and non-limiting sense.

1. A dental scope, comprising
a stretchable rod,
at least a fog-repellent reflection mirror group, comprising at least a first fog-repellent reflection mirror connected to a distal end of said stretchable rod, and at least a second fog-repellent reflection mirror connected to a primal end of said stretchable rod; and
at least a light module, comprising at least a spot light source, at least a battery and at least a switch disposed on said stretchable rod, wherein said spot light source is employed to illuminate an interior of user's mouth.

2. The dental scope according to claim 1, wherein said stretchable rod comprises a rod with a fixed length, stretchable length or bendable.

3. The dental scope according to claim 1, wherein said spot light source is positioned on a surface or a hollow inner portion of said stretchable rod.

4. The dental scope according to claim 1, wherein said spot light is positioned on said first fog-repellent reflection mirror.

5. The dental scope according to claim 1, wherein said fog-repellent reflection mirror is screwed or riveted to said stretchable rod.

6. The dental scope according to claim 1, wherein said fog-repellent reflection mirror is detachably fixed or riveted to said stretchable rod.
